(19) United States
(12) Patent Application Publication
(10) Pub. No.: US 2011/0257375 A1
(43) Pub. Date: Oct. 20, 2011

(54) INCREASING EFFICIENCY OF NUCLEIC ACID DELIVERY IN VIVO USING TARGETING CONJUGATES

(76) Inventors: Lance P. Ford, Austin, TX (US); Joe Krebs, Austin, TX (US); Brittan L. Pasloske, Austin, TX (US)

(21) Appl. No.: 12/933,792

(22) PCT Filed: Feb. 11, 2009
(86) PCT No.: PCT/US09/33783

§ 371 Date: Jul. 6, 2011

Related U.S. Application Data
(60) Provisional application No. 61/027,813, filed on Feb. 11, 2008.

(51) Int. Cl. C07K 19/00

(52) U.S. Cl. 530/391.7; 530/391.1

(57) ABSTRACT

Described herein is the use of antibody-based delivery agents to target and deliver nucleic acid agents into specific cell types. Herein, we describe methods used that improve the ability of conjugates that load over 3 siRNA per conjugate and target siRNA to cells expressing the appropriate cell surface antigens. We also contemplate the use of antibody, targeting peptides, small molecules, aptamers and all other factors known in the art that can specifically target tissues. In each case, these targeting moieties can be conjugated using chemical crosslinking agents to carriers enabling directed delivery of nucleic acids.

### BACKGROUND OF THE INVENTION

1. Field of the Invention

The present invention generally relates to methods and compositions that mediate the delivery of nucleic acids into living organisms and cells.

2. Description of the Relevant Art

The use of nucleic acids have proved effective for altering the state of a cell. The introduction of deoxyribonucleic acid (DNA) or ribonucleic acid (RNA) into a cell can be used to up- or down-regulate the expression of particular genes in the cell, thereby, impacting one or more biochemical pathways. The introduction of specific genes encoded in double-stranded DNA, as in a plasmid, leads to the expression of the encoded gene in the cell. The introduction of antisense-RNA or -DNA, ribozymes or short interfering RNA (siRNA) can be used to down-regulate specific genes targeted by these entities. There are many technologies that mediate the transfer of these nucleic acids into cells in vitro. However, the introduction of nucleic acids in vivo remains challenging.

Of the nucleic acid-based technologies used to alter cell physiology, RNA interference (RNAi) is the general term given for regulating the expression of genes at the post-transcriptional level in diversified organisms. This biological pathway has already been used for functional genomic screening, drug discovery and in vivo target validation with RNAi agents and encompasses, but is not limited to, short interfering RNA (siRNA), micro-RNA (miRNA), antisense oligonucleotides, ribozymes, aptamers, and piwi-interacting RNAs (piRNAs). Although agents used by the RNAi pathway are in active development as gene-specific medicines, the future success of RNAi technology is intricately connected to the development of non-toxic drug carriers that efficiently direct RNAi agents to desired locations within organisms and mediate the transfection of the cells in those locations, in a cost effective fashion.

A cellular pathway used by RNAi agents is depicted in FIG. 1. RNAi agents can enter this pathway inside cells such as is done for miRNA or by transfection as is done using synthetically manufactured RNAi compounds. Capped and polyadenylated miRNA transcripts called pri-miRNA are generated by RNA polymerase in the nucleus. Pri-miRNA in the nucleus are cleaved into a pre-miRNA by Drosha (DGCR8 in mammals) which exist as stem loop structures that are typically under 100 bases in length. The pre-miRNA is then exported into the cytoplasm using the exportin 5/RanGTP protein complex. In the cytoplasm the enzyme Dicer together with (tar)-binding protein cleaves the pre-miRNA liberating the mature 21-nucleotide mature duplex. One strand of the miRNA duplex preferentially enters the RNA-induced silencing complex (RISC). If 25 to 27-mer RNAi agents are used, DICER cleaves them into 21-mers with 2-nt 3′ overhangs before inclusion into the RISC while synthetic 21-mer RNAi agents enter RISC directly. The RISC involved in pairing the mature single-stranded RNAi agent with the target mRNA contains an Argonaute (Ago) protein family member and the endonuclease target cleaving activity of RISC is Ago2. Although siRNA enter Ago2 containing RISCs, miRNAs often enter RISC's that contain Ago1 which lack cleaving activity. Considering siRNA and miRNA utilize differentiated RISC, RNA mediated gene inhibition at transcriptional levels may use different cellular pathways all together. The RISC together with the small RNA and its target are delivered to cytoplasmic foci called Processing bodies (P-bodies) also known as GW bodies where post-transcriptional gene repression takes place. RNAi has also been proposed to take place in the nucleus by influencing transcription.

RNAi experiments in living organisms are challenging since cells are typically impermeable to macromolecules, including proteins and nucleic acids. The lack of effective methods for delivering macromolecules into organisms has been the obstacle to the therapeutic use of large numbers of nucleic acids having intracellular sites of action. To overcome this obstacle, many techniques have been developed to deliver macromolecules into cells. These methods include but are not limited to electroporation, membrane fusion with liposomes, high velocity bombardment, fusogenic peptides and antibody conjugates.

The approach for directing the delivery of RNAi agents in vivo with affinity reagents, especially antibodies, is promising. Over 20 antibody-based therapies are on the market to treat diseases such as breast cancer (e.g., Herceptin), inflammatory diseases (e.g., Xolair, Tysabri, Remicade, Raptiva), non-Hodgkin lymphoma (e.g., Bexxar, Rituxan, Zevalin) and transplant rejection (e.g., Orthoclone, Zenapax). Antibody-based drugs are commonly used for delivering toxic payloads to specific cell types in a therapeutic setting (eg., Mylotarg). Mylotarg binds to the cell surface receptor (CD33) and delivers the toxic payload (calicheamicin) into CD33 positive acute myeloid leukemia cells. After binding to CD33, Mylotarg is taken into acute myeloid leukemia cells through receptor-mediated endocytosis giving calicheamicin access to the intracellular environment cleaving DNA and killing the cancer cells.

### SUMMARY OF THE INVENTION

The embodiments described herein relate to the production of conjugates which are used to deliver nucleic acids to specific cells in a living organism or to cells in vitro. Furthermore, embodiments described herein apply to the use of conjugates including, but not limited to, antibodies for directing nucleic acids, more preferably, RNAi agents to specific locations in living organisms or to cells in vitro.

In one embodiment, conjugates may be made through the use of cross-linking agents to create covalent bonds between ligands and carriers. These approaches have utility for advancing the therapeutic application of RNAi agents and the ability of RNAi agents to be used for in vivo target validation.

In one embodiment, a covalent bond is formed between a carrier and a ligand to produce a conjugate. Nucleic acid is bound to the conjugate through non-covalent bonds. The conjugate with its associated nucleic acid is administered to a subject or to cells in vitro and is bound by cells that bind specifically with the ligand of the conjugate. When the cell internalizes the conjugate, the nucleic acid is released and acts by various means on the cell to alter its state.

In one embodiment, a method of treating diseases in mammals includes administering the loaded conjugate to a mammalian subject in an effective amount, the amount being sufficient to at least partially relieve some symptoms of the disease.

In some embodiments, the compositions and methods may be used to treat diseases and disorders characterized by the under-expression or over-expression of a gene or group of genes, including genes with mutations. In some aspects, the compositions and methods may be used to treat metabolic diseases and disorders (e.g., diseases and disorders in which the liver is a target such as, for example, dyslipidemia and diabetes). One of skill in the art will appreciate that silencing of genes associated with metabolic diseases and disorders can be combined with conventional treatments for disorders such as these. In another embodiment, the composition may be used in the treatment of infectious diseases including those caused by viruses, bacteria and fungi.

In some embodiments, the compositions and methods may be used to deliver nucleic acids to cells in vitro and then assay for changes in the cell, including but not limited to, at the molecular or the phenotypic level. Such experiments may be useful in characterizing the function of specific genes or identifying drug targets.

### BRIEF DESCRIPTION OF THE DRAWINGS

Other objects and advantages of the invention will become apparent upon reading the following detailed description and upon reference to the accompanying drawings in which:

FIG. 1 is a schematic representation of the induction of the RNA-interference pathway in a cell in response to various RNAi agents.

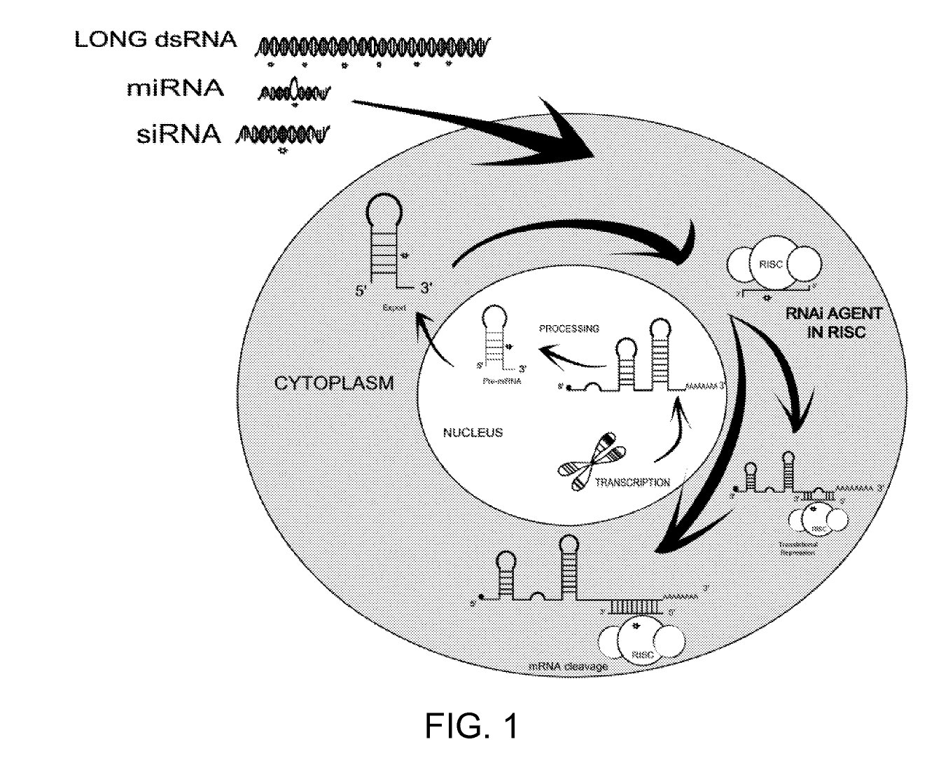

While the invention is susceptible to various modifications and alternative forms, specific embodiments thereof are shown by way of example in the drawings and will herein be described in detail. It should be understood, however that the drawing and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION

In an embodiment, a conjugate comprised of a carrier and a ligand is used to deliver nucleic acids to a living organism. In an embodiment, the carrier is chemically bonded to the ligand using cross-linking reagents to produce the conjugate. The conjugate is loaded with the nucleic acid prior to administration to the living organism.

“Ligands”, as used herein, are molecules that bind specifically to a cell surface. Usually, the ligand binds specifically to cell receptors. Ligands include, but are not limited to proteins, glycoproteins, carbohydrates, aptamers or lipids.

In one embodiment, the ligand is a monoclonal antibody (MAB). A MAB may be isolated from ascites fluid from a mouse or rat or it may be isolated from the supernatant of cells grown in culture. The MAB used as a ligand may be of the IgG subclass but may also be an IgM, IgE or IgA. Usually, an IgG MAB is affinity purified using protein A or G sepharose. In other instances, the MAB may be isolated by salt fractionation. MABs are commercially available from a variety of vendors. In some embodiments, the MAB is selected based on its ability to recognize the native structure of the cell receptor. In many instances, a MAB recognizes only a denatured form of the cell receptor and is useful for applications such as immunoblotting or fixed immunofluorescence assays (IFA). A MAB will most likely be functional if it functions in immunoprecipitations or non-fixed IFA.

In one embodiment, the ligand is a derivative of a MAB containing the complementary—determining region (CDR) or antigen binding site. In some aspects, the MAB fragment is a recombinant protein. One skilled in the art will appreciate that there are several methods to generate and modify a MAB. In some embodiments, the MAB fragment is recombinantly expressed and purified as a single chain fragment variable (scFv). In some aspects, the recombinant MAB may be genetically manipulated to be more amenable to carrier conjugation. In some aspects, a MAB may be digested with proteases to generate fragments of the MAB (Fab) that may bind to the antigen. In another embodiment, the carbohydrate residues of the MAB may be treated chemically or enzymatically to be more amenable to carrier conjugation.

In another embodiment, the ligand may be a polyclonal antibody. The IgG fraction is isolated from serum. In these instances, the Fc portion of the IgG is recognized by the Fc receptors on cells like macrophages.

In another embodiment, the ligand is the natural ligand of the cell receptor. Natural ligands are peptide hormones (e.g., insulin), growth factors (e.g., epidermal growth factor), small molecules (e.g., folate), proteins and glycoproteins (eg. orosomucoid). The ligand may also be a derivative of the natural ligand such as a fragment of the ligand which binds to the cell receptor. In another embodiment, the ligand is a recombinant protein of the natural ligand. In another embodiment, the natural ligand is modified. For example, orosomucoid, a glycoprotein recognized by liver cell, has its terminal sialic acid residues removed by mild acid hydrolysis to form asialoorosomucoid.

“Carriers”, as used herein, are molecules that bind to nucleic acids non-covalently. The binding is reversible such that the nucleic acid is released once inside of a cell where it can exert its effect.

In one embodiment, carriers may have a net positive charge at or near physiological pH (e.g., in solutions having a pH between about 4 and 10, about 5 and 9, or about 6 and 8) and bind to the nucleic acids through electrostatic interactions. Carriers of this class include, but are not limited to protamine, Sso7d, histones, poly Lysine, poly Arginine, avidin, synthetic polypeptides, non-proteinaceous molecules (e.g., non-peptide cations), and carbon nanotubes, modified to comprise a net positive charge.

“Protamines”, as used herein, refers to small, strongly basic proteins, the positively charged amino acid groups of which (especially arginines) are usually arranged in groups and neutralize the negative charges of nucleic acids because of their polycationic nature. Protamines may be of natural origin or produced by recombinant methods. Use of recombinant methods allows multiple copies of the protamine to be produced or modifications may be made in the molecular size and amino acid sequence of the protamine. Corresponding compounds may also be chemically synthesized. When an artificial protamine is synthesized, the procedure used may include, for example, replacing amino acid residues which have functions in the natural protamine that are undesirable for the transporting function (e.g., the condensation of DNA) with other suitable amino acids.

“Histones”, as used herein, refer to small DNA-binding proteins present in the chromatin having a high proportion of positively charged amino acids (lysine and arginine) which enable them to bind to DNA independently of the nucleotide sequence and fold it into nucleosomes, the arginine-rich histones H3 and H4 being particularly suitable. As for the preparation and modifications thereof, the remarks made above in relation to protamines apply here as well.

Synthetic polypeptides include peptides such as homologous polypeptides (polylysine, polyarginine) or heterologous polypeptides (that include two or more representatives of positively charged amino acids).

Non-peptide cations include polymeric cations (such as polyethyleneimines). The size of the polymeric cation is preferably selected so that the sum of the positive charges is about 10 to 500 and this is, in some embodiments, matched to the nucleic acid which is to be transported.

In another embodiment, the carrier binds specifically to a small molecule which is covalently bonded to the nucleic acid. Small molecules include, but are not limited to biotin, fluorescein isothiocyanate (FITC), and dinitrophenol (DNP). Carriers of this class include avidin and streptavidin which bind tightly to biotin. Additionally, there are MABs that bind strongly to biotin, FITC and DNP and other small molecules. In another embodiment, the carrier is a MAB derivative.

“Conjugates”, as used herein, are comprised of a ligands chemically conjugated to carriers. The conjugation is typically mediated by cross-linking agents.

In one embodiment, after purification of conjugate, the ratio between the carrier and the ligand is between 1:10 and 1:1. In another embodiment, the ratio is between 1:1 and 5:1. In another embodiment, the ratio is between 1:1 and 10:1. In an embodiment, the ratio is greater than 1:1. In another embodiment, the ratio is greater than 4:1.

In one embodiment, the cross-linking agents are hetero-bifunctional having functional groups including but not limited to, aryl azides, carbodiimide, hydrazine, imidoester, isocynate, maleimide, N-hydroxysuccinimide (NHS)-ester and sulfo-NHS-ester. The bonds they produce include, but are not limited to, amide, disulfide, hydrazone and ester bonds.

Examples of cross-linking agents include, but are not limited to, succinimidyl 4-formylbenzoate (SFB), succinimidyl 4-hydrazinonicotinate acetone hydrazone (SANH), 1-ethyl-3-[3-dimethylaminopropyl]carbodiimide (EDC), N-succinimidyl 3-(2-pyridyldithio)-propionate (SPDP), 2-Iminothiolane (Traut's Reagent), SATA, and 3[2-pyridyldithio]propionyl hydrazide (PDPH).

In one embodiment, SFB is reacted with free primary amine groups of the carrier while introducing reactive aldehyde groups. SANH is reacted with free primary amines of the ligand while introducing reactive hydrazine groups. The SFB activated-carrier is mixed with the SANH activated-ligand. The reactive aldehyde groups react with the hydrazine groups to generate hydrazone bonds, thus producing the conjugate.

In another embodiment, the SFB is reacted with free primary amines of the ligand and the SANH is reacted with free primary amines of the carrier. The activated carrier and ligand are mixed and reacted to form a conjugate through hydrazone bonds.

In one embodiment, SPDP is reacted with the free primary amine groups in separate reactions with the carrier and the ligand. Either the activated ligand or the activated carrier are then subjected to reduction using a reductant such as dithiothreitol, beta-mercaptoethanol or tris(2-carboxyethyl)phosphine (TCEP). After the reduction step, the ligand and carrier are mixed to form a conjugate through disulfide bonds.

In another embodiment, the ligand, carrier and cross-linking agent are mixed together and conjugates are formed through amide bonds.

In another embodiment, the cross-linkers are homo-bifunctional and include, but are not limited to glutaraldehyde and formaldehyde.

In one embodiment, glutaraldehyde is combined with the ligand and carrier in one reaction. The aldehyde groups of the glutaraldehyde react with primary amines on the proteins to form bonds and generate the conjugate. The disadvantage of these reactions is that ligand-ligand and carrier-carrier conjugates can be produced in addition to the desired ligand-carrier conjugate. In other embodiments, formaldehyde may be used by forming methylene bridges between proteins. Formaldehyde, however, may also generate unwanted homo-dimers and aggregates.

In one embodiment, the conjugate binds at least one nucleic acid molecule. In another embodiment, the conjugate binds at least 2 nucleic acid molecules. In another embodiment, the conjugate binds at least 3 nucleic acid molecules. In another embodiment, the conjugate binds at least 4 nucleic acid molecules.

“Nucleic acids”, as used herein, refer to polymers of deoxyribonucleotides and/or ribonucleotides to form strands of DNA, RNA or a hybrid strand of DNA and RNA, with no restrictions as to the nucleotide sequence. The nucleotides may be chemically modified; these modifications include, for example, the substitution of the phosphodiester group by phosphorothioates or the use of nucleoside analogues. The nucleic acids may be modified to include an affinity handle to promote non-covalent binding to the carrier. Examples of affinity handles include, but are not limited to, biotin, FITC and DNP. The nucleic acid can be single- or double-stranded.

In one embodiment, the nucleic acid is an RNAi agent which is used to reduce gene expression, directly or indirectly. Examples of RNAi agents include, but are not limited to, siRNA, piRNA, ribozymes, and antisense oligonucleotides.

With regard to the size of the nucleic acids the invention again permits a wide range of uses. There is no lower limit brought about by the transporting system; any lower limit which might arise would be for reasons specific to the application, e.g., because antisense oligonucleotides with less than about 10 nucleotides would, in the specific application, not be suitable owing to their lack of specificity. Using the conjugates according described herein it is also possible to convey plasmids into the cell, the smaller plasmids which are used as carrier nucleic acids (e.g., retroviral vectors) being of particular practical use. It is also possible to convey different nucleic acids into the cell at the same time using the conjugates described herein.

“Loading”, as used herein, refers to the process of binding the nucleic acid to the conjugate. Typically, the conjugate is loaded with nucleic acid by incubating the nucleic acid and conjugate together in phosphate buffered saline at about 21° C. for 15 to 30 minutes. After the incubation, the mixture can be administered directly to the subject. In another embodiment, the non-loaded nucleic acid is separated from the loaded conjugate before the loaded conjugate is administered to the cells or subject.

The ratio of nucleic acid to conjugate may vary within a wide range, and it is not absolutely necessary to neutralize all the charges of the nucleic acid. This ratio may be adjusted for each individual case depending on criteria such as the size and structure of the nucleic acid which is to be transported, the size of the carrier and the number and distribution of the carrier's charges, so as to achieve a ratio of transportability and biological activity of the nucleic acid favorable to the application.

In one embodiment, conjugates targeting different cell surface receptors may be mixed, thereby broadening the specificity of the cell types targeted. In one aspect, a VEGF-R2 conjugate may be mixed with a Her2 conjugate. In another embodiment, a conjugate may be loaded with more than one type or sequence of nucleic acid. In one aspect, a VEGF-R2 conjugate may be loaded with an siRNA directed to GAPDH and an siRNA directed to c-myc. In another embodiment, more than one conjugate may be loaded with more than one type or sequence of nucleic acid.

In one embodiment, pharmaceutical preparations that include, as the active component a nucleic acid which specifically inhibits gene expression, loaded onto a conjugate may be used to treat a subject. Such pharmaceutical preparations may be used to inhibit pathogenic viruses such as HIV or related retroviruses, oncogenes or other key genes which control growth and/or differentiation of cells, e.g., the c-fos gene or the c-myc gene. Another field of use is in fighting diseases by inhibiting the production of undesirable gene products, e.g., the major plaque protein which occurs in Alzheimer's disease or proteins which cause autoimmune diseases.

In one embodiment, the conjugate-nucleic acid compound may be formulated as a pharmaceutical composition. Any suitable route of administration may be employed for providing a patient with an effective dosage of drugs of the present invention. For example, oral, rectal, topical, parenteral, ocular, intracranial, pulmonary, nasal, and the like may be employed. Dosage forms may include tablets, troches, dispersions, suspensions, solutions, capsules, creams, ointments, oils, emulsions, liposomes, aerosols, and the like. In certain embodiments, it may be advantageous that the compositions described herein be administered orally. In other embodiments, it may be advantageous that the compositions described herein be administered parenterally. In yet other embodiments, it may be advantageous that the compositions described herein be administered locally, at the site of tissue injury.

The pharmaceutical compositions may include those compositions suitable for oral, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. They may be conveniently presented in unit dosage form and prepared by any of the methods well known in the art of pharmacy.

For administration by inhalation, the compositions described herein are conveniently delivered in the form of an aerosol spray presentation from pressurized packs or nebulisers. The compositions may also be delivered as powders which may be formulated and the powder composition may be inhaled with the aid of an insufflation powder inhaler device.

Suitable topical formulations for use in the present embodiments may include transdermal devices, aerosols, creams, ointments, lotions, dusting powders, and the like.

In practical use, compositions can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

The pharmaceutical preparations may be manufactured in a manner which is itself known to one skilled in the art, for example, by means of conventional mixing, granulating, dragee-making, softgel encapsulation, dissolving, extracting, or lyophilizing processes. Thus, pharmaceutical preparations for oral use may be obtained by combining the active compounds with solid and semi-solid excipients and suitable preservatives, and/or co-antioxidants. Optionally, the resulting mixture may be ground and processed. The resulting mixture of granules may be used, after adding suitable auxiliaries, if desired or necessary, to obtain tablets, softgels, lozenges, capsules, or dragee cores.

Suitable excipients may be fillers such as saccharides (e.g., lactose, sucrose, or mannose), sugar alcohols (e.g., mannitol or sorbitol), cellulose preparations and/or calcium phosphates (e.g., tricalcium phosphate or calcium hydrogen phosphate). In addition binders may be used such as starch paste (e.g., maize or corn starch, wheat starch, rice starch, potato starch, gelatin, tragacanth, methyl cellulose, hydroxypropylmethyl-cellulose, sodium carboxymethylcellulose, and/or polyvinyl pyrrolidone). Disintegrating agents may be added (e.g., the above-mentioned starches) as well as carboxymethyl-starch, cross-linked polyvinyl pyrrolidone, agar, or alginic acid or a salt thereof (e.g., sodium alginate). Auxiliaries are, above all, flow-regulating agents and lubricants (e.g., silica, talc, stearic acid or salts thereof, such as magnesium stearate or calcium stearate, and/or polyethylene glycol, or PEG). Dragee cores are provided with suitable coatings, which, if desired, are resistant to gastric juices. Softgelatin capsules (“softgels”) are provided with suitable coatings, which, typically, contain gelatin and/or suitable edible dye(s). Animal component-free and kosher gelatin capsules may be particularly suitable for the embodiments described herein for wide availability of usage and consumption. For this purpose, concentrated saccharide solutions may be used, which may optionally contain gum arabic, talc, polyvinyl pyrrolidone, polyethylene glycol (PEG) and/or titanium dioxide, lacquer solutions and suitable organic solvents or solvent mixtures, including dimethylsulfoxide (DMSO), tetrahydrofuran (THF), acetone, ethanol, or other suitable solvents and co-solvents. In order to produce coatings resistant to gastric juices, solutions of suitable cellulose preparations such as acetylcellulose phthalate or hydroxypropylmethyl-cellulose phthalate, may be used. Dye stuffs or pigments may be added to the tablets or dragee coatings or softgelatin capsules, for example, for identification or in order to characterize combinations of active compound doses, or to disguise the capsule contents for usage in clinical or other studies.

Other pharmaceutical preparations that may be used orally include push-fit capsules made of gelatin, as well as soft, thermally sealed capsules made of gelatin and a plasticizer such as glycerol or sorbitol. The push-fit capsules may contain the active compounds in the form of granules that may be mixed with fillers such as, for example, lactose, binders such as starches, and/or lubricants such as talc or magnesium stearate and, optionally, stabilizers and/or preservatives. In soft capsules, the active compounds may be dissolved or suspended in suitable liquids, such as fatty oils such as rice bran oil or peanut oil or palm oil, or liquid paraffin. In some embodiments, stabilizers and preservatives may be added.

In some embodiments, pulmonary administration of a pharmaceutical composition may be desirable. Pulmonary administration may include, for example, inhalation of aerosolized or nebulized liquid or solid particles of the pharmaceutically active component dispersed in and surrounded by a gas.

Possible pharmaceutical preparations, which may be used rectally or vaginally, include, for example, suppositories, which include a combination of the active compounds with a suppository base. Suitable suppository bases are, for example, natural or synthetic triglycerides, or paraffin hydrocarbons. In addition, it is also possible to use gelatin rectal capsules that consist of a combination of the active compounds with a base. Possible base materials include, for example, liquid triglycerides, polyethylene glycols, or paraffin hydrocarbons.

Suitable formulations for parenteral administration include, but are not limited to, aqueous solutions of the active compounds in water-soluble and/or water dispersible form, for example, water-soluble salts, esters, carbonates, phosphate esters or ethers, sulfates, glycoside ethers, together with spacers and/or linkers. Suspensions of the active compounds as appropriate oily injection suspensions may be administered, particularly suitable for intramuscular injection. Suitable lipophilic solvents, co-solvents (such as DMSO or ethanol), and/or vehicles including fatty oils, for example, rice bran oil or peanut oil and/or palm oil, or synthetic fatty acid esters, for example, ethyl oleate or triglycerides, may be used. Aqueous injection suspensions may contain substances that increase the viscosity of the suspension including, for example, sodium carboxymethyl cellulose, sorbitol, dextran, and/or cyclodextrins. Cyclodextrins (e.g., β-cyclodextrin) may be used specifically to increase the water solubility for parenteral injection of the compound. Liposomal formulations, in admixture with, for example, egg yolk phosphotidylcholine (E-PC), may be made for injection. Optionally, the suspension may contain stabilizers, for example, antioxidants such as BHT, and/or preservatives, such as benzyl alcohol.

The compositions of this invention can be administered in such oral dosage forms as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular form, all using dosage forms well known to those of ordinary skill in the pharmaceutical arts. They can be administered alone, but generally will be administered with a pharmaceutical carrier selected on the basis of the chosen route of administration and standard pharmaceutical practice.

The dosage regimen for the compounds of the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. A physician or veterinarian may determine and prescribe the effective amount of the drug required to prevent, counter, or arrest the progress or the development prostate cancer in a subject. The pharmaceutical compositions may be administered in a single daily dose, or the total daily dosage may be administered in divided doses of two, three, or four or more times daily.

The pharmaceutical compositions described herein may further be administered in intranasal form via topical use of suitable intranasal vehicles, or via transdermal routes, using transdermal skin patches. When administered in the form of a transdermal delivery system, the dosage administration will, of course, be continuous rather than intermittent throughout the dosage regimen.

The compounds are typically administered in admixture with suitable pharmaceutical diluents, excipients, or carriers (collectively referred to herein as “pharmacologically inert carriers”) suitably selected with respect to the intended form of administration, that is, oral tablets, capsules, elixirs, syrups and the like, and consistent with conventional pharmaceutical practices.

For instance, for oral administration in the form of a tablet or capsule, the pharmacologically active component may be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water, and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds of the present invention may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Compounds of the present invention may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water-soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol.

Suitable pharmaceutical carriers are described in Remington's Pharmaceutical Sciences, Mack Publishing Company, a standard reference text in this field.

In an embodiment, the pharmaceutical composition may be administered to the patient systemically. The term systemic as used herein includes subcutaneous injection; intravenous, intramuscular, intraestemal injection; infusion; inhalation, transdermal administration, oral administration; and intra-operative instillation.

One systemic method involves an aerosol suspension of respirable particles comprising the active compound, which the subject inhales. The active compound would be absorbed into the bloodstream via the lungs, and subsequently contact the lacrimal glands in a pharmaceutically effective amount. The respirable particles may be liquid or solid, with a particle size sufficiently small to pass through the mouth and larynx upon inhalation; in general, particles ranging from about 1 to 10 microns, but more preferably 1-5 microns, in size are considered respirable.

Another method of systemically administering the active compounds involves administering a liquid/liquid suspension in the form of eye drops or eye wash or nasal drops of a liquid formulation, or a nasal spray of respirable particles that the subject inhales. Liquid pharmaceutical compositions of the active compound for producing a nasal spray or nasal or eye drops may be prepared by combining the active compound with a suitable vehicle, such as sterile pyrogen free water or sterile saline by techniques known to those skilled in the art.

The active compounds may also be systemically administered through absorption by the skin using transdermal patches or pads. The active compounds are absorbed into the bloodstream through the skin. Plasma concentration of the active compounds can be controlled by using patches containing different concentrations of active compounds.

Other methods of systemic administration of the active compound involves oral administration, in which pharmaceutical compositions containing active compounds are in the form of tablets, lozenges, aqueous or oily suspensions, viscous gels, chewable gums, dispersible powders or granules, emulsion, hard or soft capsules, or syrups or elixirs. Additional means of systemic administration of the active compound to the subject may involve a suppository form of the active compound, such that a therapeutically effective amount of the compound reaches the eyes via systemic absorption and circulation.

Further means of systemic administration of the active compound involve direct intra-operative instillation of a gel, cream, or liquid suspension form of a therapeutically effective amount of the active compound.

For topical application, the solution containing the active compound may contain a physiologically compatible vehicle, as those skilled in the art can select, using conventional criteria. The vehicles may be selected from the known pharmaceutical vehicles which include, but are not limited to, saline solution, water polyethers such as polyethylene glycol, polyvinyls such as polyvinyl alcohol and povidone, cellulose derivatives such as methylcellulose and hydroxypropyl methylcellulose, petroleum derivatives such as mineral oil and white petrolatum, animal fats such as lanolin, polymers of acrylic acid such as carboxypolymethylene gel, vegetable fats such as peanut oil and polysaccharides such as dextrans, and glycosaminoglycans such as sodium hyaluronate and salts such as sodium chloride and potassium chloride.

For systemic administration such as injection and infusion, the pharmaceutical formulation is prepared in a sterile medium. The active ingredient, depending on the vehicle and concentration used, can either be suspended or dissolved in the vehicle. Adjuvants such as local anaesthetics, preservatives and buffering agents can also be dissolved in the vehicle. The sterile injectable preparation may be a sterile injectable solution or suspension in a non-toxic acceptable diluent or solvent. Among the acceptable vehicles and solvents that may be employed are sterile water, saline solution, or Ringer's solution.

In practical use, the conjugate-nucleic acid used can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

The following will serve to illustrate, by way of one or more examples, systems and methods for preparing conjugates and their usefulness in delivering nucleic acids to living organisms. The examples set forth below are non-limiting and are intended to be merely representative of various aspects and features of certain embodiments. Although methods and materials similar or equivalent to those described herein may be used in the application or testing of the present embodiments, suitable methods and materials are described below.

### Example 1

### Gel Shift Assay for Conjugation Analysis

In order to monitor for the successful conjugation of carrier to ligand, in this example IgG, a gel shift analysis was performed using a protein gel stained with Coomassie Blue. IgG is comprised of 2 small subunits (about 25 kDa each) and 2 large subunits (about 50 kDa each). Conjugation of another protein (such as carrier) to the subunits increases their molecular weights. A successful conjugation is signaled by the production of larger IgG subunits. To assess the efficiency of a conjugation, non-conjugated IgG is electrophoresed beside samples of conjugated IgG using an acrylamide gel containing SDS. Prior to running the gel, the samples are reduced using a reducing agent such as beta-mercaptoethanol so the IgG subunits are de-coupled. IgG subunits that have been conjugated will migrate slower in the gel as discrete bands compared to the non-conjugated reference. If one subunit completely shifts to a higher molecular weight, then both of the subunits must have been conjugated, indicating that there are at least 2 carriers per IgG. If both subunits completely shift, then there are at least 4 carriers per IgG.

### Example 2

### A. Capacity Assay Using Fluorescent siRNA

To assess the average number of siRNA bound per conjugate molecule, fluorescent siRNA are incubated with conjugate and bound siRNA versus unbound is measured. Typically 1 pmole of conjugate is mixed with 100 pmoles siRNA (Sigma, #GUAR50) in 50 μl of 1×PBS, 30 min at 21° C. The mixture was added to the YM100 Centricon unit (Millipore, #42413) and spun at 14,000 rpm for 12 minutes. The flow through containing unbound siRNA was removed and placed into a 96 well plate. An additional 90 μl of PBS was added to the filter unit to wash the bound siRNA. The filter unit was inverted and the bound material was retrieved by centrifugation. The unbound and bound fractions were quantified in the fluorescent Packard Fusion plate reader. The percent of the bound fluorescent signal was calculated from the total fluorescent signal which was then used to calculate the molar ratio of siRNA to conjugate. To assess the background fluorescence, for each siRNA binding reaction, we measured the siRNA binding capacity of unmodified antibody.

### B. Capacity Assay Using RT-PCR

Vascular Endothelial Growth Factor-Receptor 2 (VEGF-R2) or bovine serum albumin (BSA) (DCN, #GR-021) at 10 μg/mL in 1×PBS were used to coat 96-well plates for 24 hours at 4° C. The wells were washed 3× with 0.1% Tween 20 in PBS. Both VEGF-R2 conjugate and non-conjugated anti-VEGF-R2 MAB were incubated for 1 hour at 37° C. in the wells coated with the VEGF-R2 or BSA and then washed 3× with the 0.1% Tween 20 in PBS. Next, the microRNA, hsa-Mir-1 (Ambion, #17150G) at 36.4 μg/mL was incubated in each well for 1 hour at 37° C. and then was washed 3× with 0.1% Tween 20 in PBS.

The bound miRNA was extracted by adding 250 μl TRIzol to each well, pipeting up-down 10× and transferring contents to a microfuge tube. After adding 0.1 mL chloroform and shaking 15 seconds, the tubes were centrifuged 12,000 rpm, 15 minutes, ˜4° C. The aqueous fraction was added to 0.2 mL isopropanol, shaken, incubated 10 minutes at 21° C. and then centrifuged 12,000 rpm, 10 minutes. The supernantant was removed, the miRNA pellet was vortexed with 0.2 mL of ethanol and then centrifuged at 5,700 rpm, 5 minutes. The supernatant was removed and dried with a nitrogen gas stream. The pellet was rehydrated with 0.1 mL of 0.1 mM EDTA.

The miRNA concentration of the samples was determined using the TaqMan® MicroRNA Reverse Transcription kit (Applied Biosystems, #4366596). The water, dNTP mix, RTscript, 10× buffer and RNase Inhibitor were combined for the required number of reactions and 7 μl was dispensed per sample followed by 5 μl of RNA sample and 3 μl of Mir-1 RT primer (Applied Biosystems, #4383444). The reaction was incubated 30 sec, 16° C.; 30 sec, 42° C.; 5 sec, 85° C. and then held at 4° C. until PCR. Water and UNG master mix were combined for the required number of reactions, except that double the recommended reaction volume was used. Add 40.85 μl of master mix per sample, 2.5 μl Real Time primer from kit, and then add 6.65 μl reverse transcription product from previous step. The samples were cycled as follows using a Bio-Rad IQ5 real time machine: 1×—95° C./5 min; 35×—(95° C./15″; 48° C./15″; 72° C./15″).

### Example 3

### Sso7d Carrier Production, Purification and Conjugation to IgG

Sso7d is a positively charged DNA binding protein that is a good candidate to be a carrier. Sso7d was expressed and purified in Escherichia coli (E. coli). A pET16b vector with the Sso7d designated sequence verified insert was inoculated from a single colony into 100 mL culture. After the cells reached log phase, a 10 mL sample was removed and the rest of the cells were induced with IPTG. After 2 and 4 hours of induction, the cells were lysed and run on a 10% polyacrylamide gel containing SDS. Sso7d was visibly induced as detected by Coomassie Blue staining.

Three liters of bacteria containing the Sso7d expression vector were IPTG induced and were harvested by centrifugation. The protein was purified from the bacteria using a Talon and HiTrap SP column (GE Healthcare, #28-9343-88). The gel shows the fractions of proteins obtained after the SP column.

SANH was reacted with Sso7d at three different molar ratios (5:1, 10:1 or 20:1). Bovine IgG (Bio-Rad, #500-0005) was reacted with SFB at a 1:6 molar ratio. The Sso7d and IgG reactions were desalted. The activated IgG was reacted with the activated Sso7d samples at a 1:32 molar ratio at 21° C., 1.5 hours followed by 4° C., 3 hours. As indicated by the gel shift assay, the conjugation with Sso7d was successful in generating conjugate.

### Example 4

### Conjugating Antibodies to Different Carriers

Chemical conjugation was performed using SANH (KPL, #80-01-04) and SFB (KPL, #80-02-04) cross-linking agents. We dissolved 60 mg of protamine (Sigma, # P4505), P19 (New England Biolabs, #M0310), Histone H1.4 (Sigma, #H4380), poly-L-Arginine (Sigma, #P7762), poly-L-Lysine (Sigma, #P2658) in 3 mL of 1× phosphate buffered saline (PBS) pH 7.5 (137 mM NaCl, 2.7 mM KCl, 10 mM Na₂HPO₄ and 2 mM KH₂PO₄). We diluted bovine IgG to 1.5 mg/mL in PBS pH 7.5 (50 mM NaH₂PO₄ and 137 mM NaCl). Protamine, P19, Histone H1.4 and MABs were dialyzed in separate bags or cassettes for three days changing buffer each day against one liter of PBS pH 7.5 at 4° C. while poly-L-Lysine was dissolved into phosphate buffer and used directly. Poly-L-Arginine was diluted in water and used directly in the conjugation reaction. After three days of dialysis, the proteins were transferred into clean polypropylene tubes. We dissolved 5 mg SANH in 0.25 mL DMF to make a 20 mg/mL (4.2 mg or 15 μmoles) stock and added 210 μl SANH to the 0.3 mL dialyzed protamine (20 mg/mL-6 mg or 1.5 μmoles) into 2.7 mL PBS pH 7.5 mixed to generate a 10:1 molar ratio of SANH to carrier and incubated at 21° C. for 2 hours. We dissolved 5 mg SFB in 0.5 mL DMF and added 4 μl 10 mg/mL SFB (40 μg or 166 nmoles) to 2.5 mL dialyzed antibodies (1.5 mg/ml-3.75 mg or 25 nmoles) to generate a 6:1 molar ratio of SFB to antibody and incubated at 21° C. for 2 hours. We then equilibrated PD10 desalting columns (GE Healthcare, #17-0851-01) with 25 mL 1×PBS, loaded 2.5 mL of each reaction onto the equilibrated column and eluted protein from column using 3.5 mL 1×PBS. We diluted 2 mL desalted carrier protein (1.71 mg/mL-3.42 mg-750 nmoles) into 2 mL 100 mM NaH₂PO₄ and 300 mM NaCl pH 6.0 buffer and added desalted antibody resulting in a 1 to 32 molar ratio between antibody and carrier. This mixture was incubated for 90 minutes at 21° C. followed by 4° C. for 3 hours and dialyzed overnight against 1 liter 1×PBS at 4° C. Samples from these reactions were fractionated on a 10% acrylamide gel with SDS to analyze reaction products and conjugation efficiency. In the case of all the antibodies and carriers conjugated in this experiment, there was a shift to a higher molecular weight for both the small and large IgG subunits indicating that the carriers had been covalently bonded to the IgG.

### Example 5

### Analysis of the Mouse Anti-VEGF-R2 MAB—Protamine Conjugate

A VEGF-R2 peptide comprising the sequence ASEELKTLEDRTK derived from the VEGF-R2 protein sequence was used to immunize mice. One MAB cross-reacted with this peptide by ELISA. 96-well plates were either coated with 100 ng/mL of VEGF-R2 peptide or PBS (negative control) for 20 hours at 4° C. The plates were washed, blocked with 1% BSA before anti-VEGF-R2 MAB was incubated in the wells of the peptide or BSA coated plates for one hour at 21° C. The plates were washed with ELISA wash buffer (PBS pH 7.2 with 0.01% Tween 20). Horseradish peroxidase (HRP)-conjugated anti-mouse secondary antibody (Sigma, #A4416) was then added into the well and incubated for one hour at 21° C. Each well was washed with ELISA wash buffer and 3, 3′,5,5′-tetramethylbenzidine was added into the wells for 15 minutes. After 15 minutes, ELISA stop buffer (2 M H₂SO₄) was added and the plate was read at OD 450 nm. The MAB reacted specifically with the VEGF-R2 peptide but not with the negative control (PBS coated) wells.

The anti-VEGF-R2 MAB also reacted with a protein by immunoblotting having the same mobility as VEGF-R2 from lysed, VEGF-R2 positive Human Umbilical Vein Endothelial Cells (HUVEC). There was no reactive protein detected from the VEGF-R2 negative HT-29 cells.

Both protamine and anti-VEGF-R2 MAB were dialyzed against PBS pH 7.5. Protamine was mixed with SANH while anti-VEGF-R2 MAB was mixed with SFB and each incubated at 21° C. for two hours. The proteins were purified using a G-75 gel filtration column (GE Healthcare, #17-0050-01) and dialyzed into 100 mM Phosphate NaCl pH 6.0 buffer. Desalted protamine (2 mL-1.71 mg/mL-3.42 mg-750 nmoles) was added to 2 mL 100 mM Phosphate NaCl pH 6.0 buffer, and then added to 3.3 mL desalted antibody (1.0 mg/mL-3.3 mg-23 nmoles). The reaction was incubated 90 minutes at 21° C. then at 4° C. for 3 hours. The ratio of protamine to MAB was 32:1. The purified fractions were run on an 8% acrylamide gel with SDS and stained using Coomassie Blue. Discrete bands with mobilities slower than the small and the large IgG subunits were observed in the gel. By a visual estimate, the slower proteins comprised ˜30% of the total IgG indicating that protamine had been successfully conjugated to about 30% of the small and large subunits.

Using the Fluorescent Capacity Assay, the ratio of bound siRNA to VEGF-R2 conjugate was determined to be ˜50 siRNA per conjugate.

### Example 6

### Optimizing Buffer Conditions for Conjugation

We added 90 μl of herring sperm protamine (Sigma, #P4505) (dialyzed in one of 4 activation buffers; TABLE 1) with 21 μl of dissolved SANH and incubated for 2 hours at 21° C. We next mixed 250 μl of bovine IgG (dialyzed in one of 4 different activation buffers; TABLE 1) with 0.4 μl of dissolved SFB and incubated for 2 hours at 21° C. The reactions were desalted using a PD10 column. We added 8 μl of desalted protamine with 10 μl of desalted antibody and incubated the reaction mixture at 4° C. for 22 hours in one of 4 conjugation buffers (TABLE 1). The samples were dialyzed into 1×PBS and analyzed using the siRNA binding assay and the gel shift assay. In all the buffers tested, 100% of the antibody was conjugated to protamine. Buffer set 4 generated the highest siRNA binding capacity. The use of different conjugation buffers resulted in conjugates with binding capacities ranging from 5.7 to 7 siRNA per conjugate. Subsequent experiments resulted in binding capacities up to 72 siRNA per conjugate which constitute increases over the previously cited capacity of no more than 3 siRNA per conjugate.

TABLE 1
Composition of Conjugation and Activation Buffers

| Buffer | | |
|---|---|---|
| Set | Conjugation Buffer | Activation Buffer |
| 1 | 50 mM NaH2PO4; 100 mM | 100 mM NaH2PO4; 300 mM |
| | NaCl (pH 7.5) | NaCl (pH 6.0) |
| 2 | 100 mM NaH2PO4; 150 mM | 100 mM NaH2PO4; 150 mM |
| | NaCl (pH 7.2) | NaCl (pH 6.0) |
| 3 | 100 mM NaH2PO4; 150 mM | 10 mM NaH2PO4; 150 mM |
| | NaCl (ph 7.2) | NaCl (pH 6.0) |
| 4 | 100 mM NaH2PO4; 150 mM | 100 mM MES; 150 mM |
| | NaCl (pH 7.2) | NaCl (pH 4.7) |

### Example 7

### Optimizing Cross-Linker Conditions for Conjugation

To optimize the conjugation of the ligand to the carrier, we tested different ratios of SFB to antibody, ranging from 6:1 to 40:1. We also tested increasing reaction times. We first prepared bovine IgG (0.89 mg/mL), SFB (5 mg dissolved in 500 μl DMF), 6 mg/mL protamine and 210 μl of SANH (5 mg in 250 μl DMF). We next mixed SFB with bovine IgG at molar ratios of 6:1, 20:1 and 40:1 and incubated 21° C. for 30 minutes, 2 hours or 4 hours. We then added the sample to an equilibrated minitrap GE desalting column. We discarded the flowthrough and collected the eluted material with 1×PBS.

To activate protamine, 0.3 mL dialyzed protamine (20 mg/mL-6 mg or 1.5 μmoles) was added to into 2.7 mL phosphate saline buffer pH 7.5, mixed and then added to 210 μl of 20 mg/mL SANH (4.2 mg or 15 μmoles). The reaction was incubated at 21° C. for 2 hours. The activated protamine was desalted in 3.5 mL of 1×PBS using a PD10 column.

The activated protamine was mixed with each of the activated IgG samples in PBS (pH 6.0) and incubated at 21° C. for 1 hour. We analyzed 40 μl of each conjugation reaction by the gel shift assay. The 40:1 SFB to antibody ratio conjugated for 2 hours, resulted in both small and large subunits migrating as higher molecular weight entities. Since an IgG is comprised of 2 small subunits and 2 large subunits, this data indicates that there were at least 4 protamine molecules per IgG. Any further increase in the ratio of SFB to antibody (60:1, 80:1 and 100:1) had no further benefit in the conjugation efficiency.

### Example 8

### Conjugate Activity In Vitro

We added 50 ng of Cy3-labeled siRNA (IDT, #Gapdh-2.2 M37280715) with 300 ng of anti-Human Epidermal Growth Factor Receptor 2 (HER-2) MAB or anti-VEGF-R2 MAB conjugated with protamine or the same MAB lacking protamine in 100 μl of 1×PBS. This mixture was incubated for 30 minutes at 21° C. and then added to 10,000 SKBR3 (HER-2 positive cell line) or HUVEC (VEGF-R2 positive cell line) cells plated onto a 48 well plate on poly lysine coated sterile cover slips. Seventy-two hours later the cells were washed with 1×PBS, fixed using 3% paraformaldyhyde, and mounted using VECTASHIELD® containing DAPI (Vector Laboratories, #H-1200). The uptake of Cy3-labeled siRNA was detected using a fluorescent microscope. The Cy3-labeled siRNA was detected in the cells exposed to the conjugates but it was not detected in the cells exposed to the MAB alone, indicating that the conjugate mediated the transfer of the siRNA into the cells. A 50× magnification of the cells showed an even distribution of cytoplasmic Cy3 siRNA indicating that siRNA is being released from the conjugates.

### Example 9

### Delivery of GAPDH siRNA into HUVEC Reduced GAPDH Expression

We added 37.5, 50, 100, 250, 500 or 750 ng of a GAPDH siRNA (Sigma, #GUAR50, 1212719) or a negative control siRNA (Sigma, #3001177869-1) with 300 or 700 ng of a VEGF-R2 conjugate comprised of anti-VEGF-R2 MAB conjugated with protamine in 100 μl of 1×PBS as prepared in Example 5. This mixture was incubated for 30 minutes at 21° C. and added to 10,000 HUVEC plated onto a 96 well plate. Seventy-two hours later, the cells were lysed using the protein lysis solution in the GAPDH ELISA kit (Bioo Scientific, #3401). GAPDH protein concentration was assayed by GAPDH ELISA according to the manual (Bioo Scientific, #3401). Compared to the negative siRNA control, GAPDH expression was reduced 60 to 80% in the HUVEC cells.

### Example 10

### Delivery of GAPDH siRNA into Mice Reduced GAPDH Expression

We analyzed the efficacy of conjugates in mice using a xenograft model system. We mixed 50 μg GAPDH siRNA (Sigma, #121719) or a negative control siRNA (Sigma, #3001177869-1) with 100 μg of Her-2 conjugate (anti-Her-2 MAB conjugated and protamine conjugated using the same procedure as in Example 5 for VEGF-R2 conjugate) in 100 μl of 1×PBS. This mixture was incubated for 30 minutes at 21° C. and then injected directly into xenograft tumors made using either human SKBR3 (high levels HER2) or MCF7 (low levels of HER2) cell lines. Seventy-two hours following the injection, the animals were sacrificed; tumors were removed and analyzed using the GAPDH ELISA according to the manufacturer's manual (Bioo Scientific, #3401). Compared to the negative control siRNA, GAPDH protein levels were reduced by 80%.

### Example 11

### A Conjugation Kit

A kit to enable the preparation of conjugates is presented. The kit would include a carrier which has been activated with a cross-linking agent including, but not limited to, SFB, SANH, and SPDP. In one embodiment, the carrier is protamine. In another embodiment, the carrier is a synthesized peptide of poly Arginine as a polymer of 8 or more amino acids. In another embodiment, the carrier is avidin or streptavidin. The kit would include a cross-linking agent used to activate the ligand. In one embodiment, the ligand is a MAB or a MAB derivative. In another embodiment, the ligand is a natural receptor or a natural receptor derivative. The kit may include an activation buffer for activating the ligand with the cross-linking agent. The kit may include a conjugation buffer to perform the conjugation of the activated ligand and activated carrier.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention may be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description to the invention. Changes may be made in the elements described herein without departing from the spirit and scope of the invention as described in the following claims. In addition, it is to be understood that features described herein independently may, in certain embodiments, be combined.

1. A method for producing a conjugate for the delivery of a nucleic acid to cells comprising:
obtaining a carrier, wherein the carrier is a protein or polypeptide having a net positive charge at or near physiological pH, and the carrier is capable of binding to the nucleic acid non-covalently;
obtaining an antibody, wherein the antibody is capable of binding to a surface of a cell; and
admixing the carrier and the antibody under conditions such that a covalent bond is created between the carrier and the antibody, wherein the molar ratio between the carrier and the antibody is at least 2.

2-3. (canceled)

4. The method of claim 1, wherein the molar ratio between the carrier and the antibody is greater than 3.

5-6. (canceled)

7. The method of claim 1, wherein the antibody is a polyclonal antibody.

8-9. (canceled)

10. The method of claim 1, wherein the antibody is a monoclonal antibody.

11-14. (canceled)

15. The method of claim 1, wherein the covalent bond between the antibody and the carrier is a hydrazone bond.

16. The method of claim 1, wherein the carrier is a protamine, a poly arginine, or a poly lysine.

17-25. (canceled)

26. A conjugate for the delivery of a nucleic acid to cells comprising:
a plurality of carriers, wherein the carriers are a protein or polypeptide having a net positive charge at or near physiological pH, and wherein the carriers are capable of binding to the nucleic acid non-covalently;
an antibody, wherein the antibody is capable of binding to a surface of a cell; and
wherein at least two or more carriers are covalently linked to the antibody.

27. The conjugate of claim 26, wherein at least three carriers are covalently linked to the antibody.

28-31. (canceled)

32. The conjugate of claim 26, wherein the antibody is a polyclonal antibody.

33-34. (canceled)

35. The conjugate of claim 26, wherein antibody is a monoclonal antibody.

36-39. (canceled)

40. The conjugate of claim 26, wherein the covalent bond between the antibody and the carrier is a hydrazone bond.

41. The conjugate of claim 26, wherein the carrier is a protamine, a poly arginine, or a poly lysine.

42-50. (canceled)

51. A method for producing a composition for the delivery of a nucleic acid to cells comprising:
obtaining a carrier, wherein the carrier is a protein or polypeptide having a net positive charge at or near physiological pH, and the carrier is capable of binding to the nucleic acid non-covalently;
obtaining an antibody, wherein the antibody is capable of binding to a surface of a cell;
admixing the carrier and the antibody under conditions such that a covalent bond is created between the carrier and the antibody to produce a conjugate, wherein the molar ratio between the carrier and the antibody is at least 2; and
mixing the conjugate with the nucleic acid, wherein the conjugate binds to the nucleic acid non-covalently.

52-72. (canceled)

73. The method of claim 51, wherein the nucleic acid is an RNAi agent.

74-77. (canceled)

78. A compound for the delivery of a nucleic acid to cells comprising:
a carrier, wherein the carrier is a protein or polypeptide having a net positive charge at or near physiological pH, and the carrier is capable of binding to the nucleic acid non-covalently;
an antibody, wherein the antibody is capable of binding to a surface of a cell; and
a nucleic acid
wherein the carrier and the antibody are covalently coupled to each other to produce a conjugate, and wherein the conjugate binds to the nucleic acid non-covalently.

79-172. (canceled)

173. The method of claim 1, wherein admixing the carrier and the antibody under conditions such that a covalent bond is created between the carrier and the antibody comprises:
mixing either the carrier or the antibody with succinimidyl 4-formylbenzoate (SFB) to produce an activated carrier or an activated antibody;
mixing either the carrier or the antibody with 4-hydrazinonicotinate acetone hydrazone (SANH) to produce an activated carrier or an activated antibody, wherein the carrier is reacted with SANH if the antibody is reacted with SFB, or the antibody is reacted with SANH if the carrier is reacted with SFB;
mixing the activated carrier with the activated antibody to produce a covalent hydrazone bond between the carrier and the antibody.

174. The method of claim 1, wherein admixing the carrier and the antibody under conditions such that a covalent bond is created between the carrier and the antibody comprises:
mixing either the carrier and the antibody with any one of: N-succinimidyl 3-(2-pyridyldithio)-propionate (SPDP), 2-Iminothiolane (Traut's Reagent), N-Succinimidyl S-Acetylthio acetate (SATA), or 3[2-pyridyldithio]propionyl hydrazide (PDPH) to produce an activated carrier and an activated antibody;
mixing either the activated carrier or the activated antibody with a reducing agent to produce a reduced activated carrier or a reduced activated antibody;
mixing either the reduced activating carrier with the activated antibody or a reduced activated antibody with the activated carrier to produce a covalent disulfide bond between the carrier and the antibody.
